(19) United States
(12) Patent Application Publication
(10) Pub. No.: US 2024/0033462 A1
(43) Pub. Date: Feb. 1, 2024

(54) Side Port For Addition Of Multiple Drugs To A Nebulizer

(71) Applicants: THE RESEARCH FOUNDATION FOR THE STATE UNIVERSITY OF NEW YORK, Albany, NY (US); InspiRx, Inc., Durham, NC (US)

(72) Inventors: Gerald SMALDONE, Stony Brook, NY (US); Rohinton TODDYWALA, Durham, NC (US); Akanksha HANDE, Durham, NC (US)

(21) Appl. No.: 18/268,555

(22) PCT Filed: Dec. 21, 2021
(86) PCT No.: PCT/US21/64554

§ 371 Date: Jun. 20, 2023

Related U.S. Application Data
(60) Provisional application No. 63/128,618, filed on Dec. 21, 2020.

(51) Int. Cl. A61M 16/08; A61M 16/14; A61M 11/06

(52) U.S. Cl. CPC A61M 16/0816; A61M 16/14; A61M 16/0883; A61M 11/06

(57) ABSTRACT

A port assembly for the addition of more than one drug solution to a jet nebulizer without disassembling the nebulizer or disconnecting the nebulizer from a ventilation circuit is provided. In an embodiment the port assembly has a sleeve with a branch and a compression nut adapted to mate with a port adapter of the nebulizer to form a channel for adding a drug solution to a nebulizer. A tube through the channel allows for the addition of a first drug. The branch on the sleeve provides a port and channel for the addition of a second solution into the nebulizer. This port assembly allows a clinician to administer a plurality of drugs to a patient using a nebulizer on a mechanical ventilator without interrupting the integrity of the breathing circuit to add an additional drug solution to the nebulizer.

### FIELD OF THE INVENTION

A port is provided on a nebulizer for the addition of a plurality of drug solutions to a nebulizer without disassembling the nebulizer or interrupting the flow of drug.

### BACKGROUND

The administration of nebulized drugs to patients on a mechanical ventilator is an important medical need. Challenges in the administration of nebulized drugs to patients on a medical ventilator include maximizing efficient delivery of the drug to the lungs of the patient and provision of properly humidified breathing gases. Inefficient drug delivery wastes drug product, which may be expensive, may cause unpredictable dosing to patients (too much or too little drug). As used herein, the term “nebulized” is also referred to as “atomized” or “aerosolized,” and all three terms are interchangeable.

In many instances more than one drug needs to be delivered simultaneously to a patient. Sometimes, one or more additional drugs need to be delivered at scheduled times without interrupting the delivery of another drug. It may be desirable to provide for the addition of multiple drugs to a nebulizer without requiring that the nebulizer be disassembled. Additionally, leaving the nebulizer intact during the addition of drugs may be desirable when the nebulizer is part of a ventilator breathing circuit wherein disassembling the nebulizer could involve interrupting the flow of breathing gases to a patient connected to a mechanical ventilator. Patients are only placed on mechanical ventilation if they have difficulty breathing on their own, so interrupting the operation of the ventilator even momentarily is undesirable. Prior art nebulizers such as disclosed in U.S. Patent Publication US 2015/0224278 A1, U.S. Pat. Nos. 5,355,872 and 8,561,607 B2 do not disclose this combination of features.

### SUMMARY OF THE INVENTION

This disclosure provides a novel port assembly for the addition of more than one drug solution to a jet nebulizer having a drug reservoir and a vertical orientation. This input port allows a clinician to administer a plurality of drugs to a patient using a nebulizer on a breathing circuit with a mechanical ventilator without interrupting the integrity of the breathing circuit to add an additional drug solution to the nebulizer.

In an embodiment, a port assembly (130) is provided for the addition of more than one drug solution to a nebulizer (10) having a drug reservoir (30), wherein the nebulizer is used to administer drugs to a patient by inhalation, and the nebulizer is part of a breathing circuit connected a mechanical ventilator.

The port assembly may include a sleeve (140) having a branch (150), wherein the sleeve has a compression nut (142) at a proximal end adapted to mate with a male threaded port (15) integral with the body of nebulizer (10), wherein the port (15) is adapted as a channel for adding a drug solution to a nebulizer, wherein the male threaded port is on an upper portion of a nebulizer such that a liquid added to the nebulizer through the port will cascade into a drug solution reservoir (30) in the nebulizer.

The sleeve (140) may have a sealed distal end (116) and a tube (120) traversing longitudinally through the seal from the distal end to the proximal end, wherein a first drug solution can be added into the nebulizer through the tube (120) without disassembling the nebulizer or the compression nut on the sleeve.

The branch (150) on the sleeve defines a channel (152) at an approximately 45° angle with respect to the distal end of an axis defined by tube (120), and the channel provides for the addition of an additional drug solution into the nebulizer.

The branch may have a cap (160) having an opening (162) therein permitting the addition of the additional drug solution to the nebulizer, wherein the additional drug solution is added by syringe or respule, wherein a plug (164) is provided to prevent air leakage from the opening (162) when the opening is sealed and not in use to add an additional drug solution, and wherein a first drug solution and an additional drug solution can be added simultaneously to the nebulizer.

In an embodiment, the plug of the port assembly of is on a tether affixed to the nebulizer such that the plug remains attached to the nebulizer when the plug is removed to expose the opening in the cap for the addition of a drug solution.

In an embodiment, the port assembly has a sleeve with a branch and a compression nut adapted to mate with a male threaded port adapted as a channel for adding a drug solution to a nebulizer, which male threaded port is on an upper portion of a nebulizer, which allows for a liquid added to the nebulizer through the port to cascade into a drug solution reservoir in the nebulizer. The branch on the sleeve defines a channel at an approximately 45° (±10°) angle with respect to the distal end, and the channel provides for the addition of a second solution into the nebulizer. The branch has a cap with an opening permitting the addition of an additional drug solution added by syringe or respule to the nebulizer. A plug is provided to prevent air leakage from the opening when the opening is not being used to add an additional drug solution to the nebulizer. The plug is on a tether, which is affixed to the nebulizer in order for the plug to remain attached to the nebulizer when the plug is removed for the addition of a drug solution.

In an embodiment an axis through the center of the branch channel is oriented at a 30° to 80° angle from a vertical axis though the center of the nebulizer.

In an embodiment, a nebulizer is provided for the administration of a nebulized drug to a patient on a mechanical ventilator, wherein the nebulizer is part of a breathing circuit. The nebulizer may have a drug input port allowing a drug solution to be added to the nebulizer without disassembling the nebulizer and without interrupting the breathing circuit, and wherein the input port has male threads.

In an alternative embodiment, a port assembly is provided for the addition of a drug solution to a nebulizer having a drug reservoir. The port assembly of this embodiment has a cap with a sealed distal end and a tube traversing longitudinally through the seal from the distal end to a proximal end, wherein a drug solution can be added into the nebulizer through the tube without disassembling the nebulizer, wherein the cap fits over a port adapted as a channel for adding a drug solution to a nebulizer such that a liquid added to the nebulizer through the port will cascade into a drug solution reservoir in the nebulizer.

In an alternative embodiment, a cap assembly is provided for the addition of a drug solution to a nebulizer having a drug reservoir and a vertical orientation, comprising a cap having an opening therein permitting the addition of an additional drug solution added by syringe or respule to the nebulizer, wherein a plug is provided to prevent air leakage from the opening when the opening is not in use to add an additional drug solution, wherein the cap is connected to the nebulizer body with a tether.

### DESCRIPTION OF THE DRAWINGS

FIG. 1A is a perspective view of a breath-enhanced jet nebulizer.

FIG. 1B is a cross-section of the nebulizer as shown in FIG. 1A.

FIG. 2 is a schematic of a breathing circuit.

FIG. 3 is an elevation view of a breath-enhanced jet nebulizer as used in this invention.

FIG. 4 is a perspective view of an embodiment of a cap that fits over nebulizer port 12.

FIG. 5A is a perspective view of an embodiment of a cap assembly that fits over port 12 for the addition of a plurality of drug solutions to the nebulizer.

FIG. 5B is an elevation view of the cap assembly from FIG. 5A.

FIG. 5C is a cross section of the cap assembly from FIGS. 5A and 5B.

FIG. 6A is a perspective view of a nebulizer with the inventive port assembly.

FIG. 6B is an elevation view showing the inventive port assembly attached to the nebulizer.

FIG. 6C is a cross section view of the nebulizer in FIG. 6A showing the internal arrangement of the port assembly allowing drugs added through the port assembly to cascade down to reservoir 30.

FIG. 6D is an alternative cross-section view of the nebulizer in FIG. 6A showing the alignment of an axis B-B′ through channel 152 and A-A′ through a central axis of the nebulizer body.

FIG. 7 is a perspective view of the tether and plug assembly.

FIG. 8A is a perspective view of the cap assembly attached to the port assembly and attached to the nebulizer, with the plug 164 disengaged.

FIG. 8B is a perspective view of the cap assembly attached to the port assembly and attached to the nebulizer, with the plug 164 engaged.

FIG. 9A is a perspective view of an alternative embodiment with cap assembly 161 directly connected to the nebulizer via tether 172.

FIG. 9B is a perspective view showing the alternative embodiment in position to accept addition of drug to the nebulizer.

FIG. 9C is a perspective view showing the alternative embodiment during normal operation of the nebulizer, with port 162 sealed.

FIG. 10 shows a plot of drug delivery from a breath enhanced nebulizer using the inventive port 130. The nebulizer with a steady state infusion of drug (initial fill 2 mL then 10 mL per hour) at t=0 to 30 mins demonstrated a linear drug delivery rate. Injection of a 3 mL bolus through the side port (an additional drug solution) while the 10 mL/hr infusion proceeded showed a linear rate with a significantly steeper slope, until the bolus was consumed at t=60. Then the linear rate of drug delivery from the continuous infusion resumed due to the steady state infusion. This demonstrates that multiple drugs can be added to the nebulizer with the inventive port while it is in-line in a breathing circuit and linear nebulization rates are obtained.

### DETAILED DESCRIPTION

Disclosed herein is a port assembly for the addition of more than one drug solution to a jet nebulizer having a drug reservoir and a vertical orientation, for the administration of one or more nebulized drugs to a patient requiring mechanical ventilation and in need of such one or more drugs. This invention provides for the addition of multiple drugs to the nebulizer without requiring that the nebulizer be disassembled. A method, such as disclosed herein, for adding a one or more drugs to a nebulizer without requiring disassembly of the nebulizer may be advantageous because drugs can be added continuously to a nebulizer without interrupting the flow of air or other breathing gases to the patient. Additionally, the inventive method allows drugs to be added continuously or over an extended period such as hours or days to the nebulizer while keeping the breathing circuit intact and delivering breathing gases to the patient at all times without interruption. As used herein, the term “nebulization” is synonymous with “aerosolization” or “atomization.”

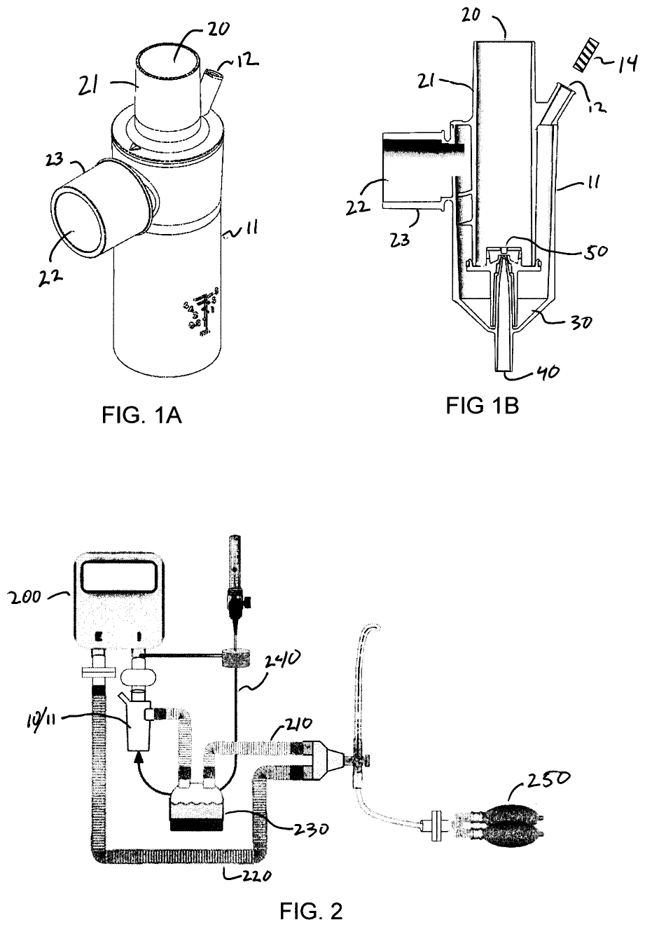

An example of a jet nebulizer is that disclosed in WO2019/236896 A1, published 12 Dec. 2019. A nebulizer (11) from WO2019/236896 is shown in in FIG. 1A (external view) and FIG. 1B (cutaway view). In an embodiment, this style of nebulizer is part of a breathing circuit for use with a mechanical ventilator (see e.g., FIG. 2). The nebulizer (10 or 11) would be on the inspiratory limb (210) of such a breathing circuit having a mechanical ventilator 200 and an expiratory limb 220. Also depicted is humidifier 230 and patient lungs 250. An exemplary mechanical ventilator for this arrangement is the “AVEA™ CVS Ventilation System.”

In the operation of the nebulizer, breathing gases from ventilator 200 enter the nebulizer at input airway 20 and exit the nebulizer, with or without nebulized drug, at output airway 22. A drug solution is held in a reservoir 30 for nebulization. Nebulization occurs when compressed air (240) is supplied to compressed air input port 40 (typically 2-6 L/min at 50 psig) which causes a Venturi effect from an air jet in Venturi section 50 that draws liquid from reservoir 30 into the Venturi where the liquid is nebulized by shear forces in the Venturi section (50). The Venturi effect and internal operation of a nebulizer such as used in this invention is explained in further detail in WO2019/236896. The nebulizer is termed a “jet nebulizer” because of the air jet.

If no compressed air is supplied to air input port 40, nebulization does not occur. Breathing gases can flow through the nebulizer regardless of whether nebulization is active or not. In an embodiment described in WO2019/236896, this nebulizer may be used in a breath actuated mode, in which a pressure sensor on a mechanical ventilation breathing circuit detects when an actual inhalation is occurring, as opposed to portions of the breathing cycle where the patient is exhaling or neither inhaling nor exhaling. In breath actuation mode, the compressed air input port 40 is toggled on only when the patient is inhaling. Thus, nebulization only occurs when the patient is actually inhaling. The jet nebulizer 11 as disclosed in WO2019/236896 is also termed a “breath enhanced” nebulizer, having an internal configuration that amplifies the Venturi effect and rate of nebulization of the drug solution.

Because of the arrangement of the reservoir, the Venturi section, and other internal features of nebulizers 10/11, these nebulizers are intended to be used in a generally vertical orientation as shown the drawings, with the input airway channel 21 on the top, and reservoir 30 on the bottom of nebulizer 10/11.

Nebulizer 11 may include a drug input port 12, that provides a means to add a drug solution to the nebulizer without the need to disassemble the nebulizer or interrupt a breathing circuit, even momentarily. In embodiments, the nebulizer may be a permanent part of a breathing circuit, meaning that once a patient is set up with a breathing circuit, the nebulizer is not removed for the entire duration of the treatment. When a drug solution is added through drug input port 12, the liquid cascades down to drug solution reservoir 30, where it is ready for nebulization.

Drug input port 12 may be further provided with plug 14 in FIG. 1B. By removing plug 14 (as shown in FIG. 1B), additional drug can be added to reservoir 30 in the exemplary jet nebulizer. When plug 14 is inserted into port 12 (not shown), it makes an airtight seal for normal operation when a drug solution is not being actively added to reservoir 30.

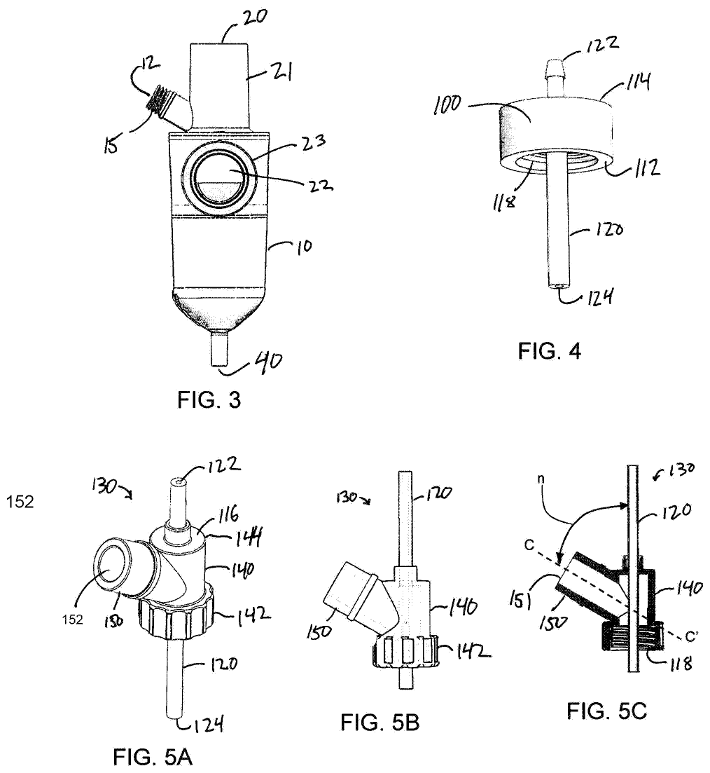

Another embodiment (10) of a jet nebulizer is shown in FIG. 3, which is similar to jet nebulizer 10, but port 12 includes male threads 15, for use with the input ports as described herein.

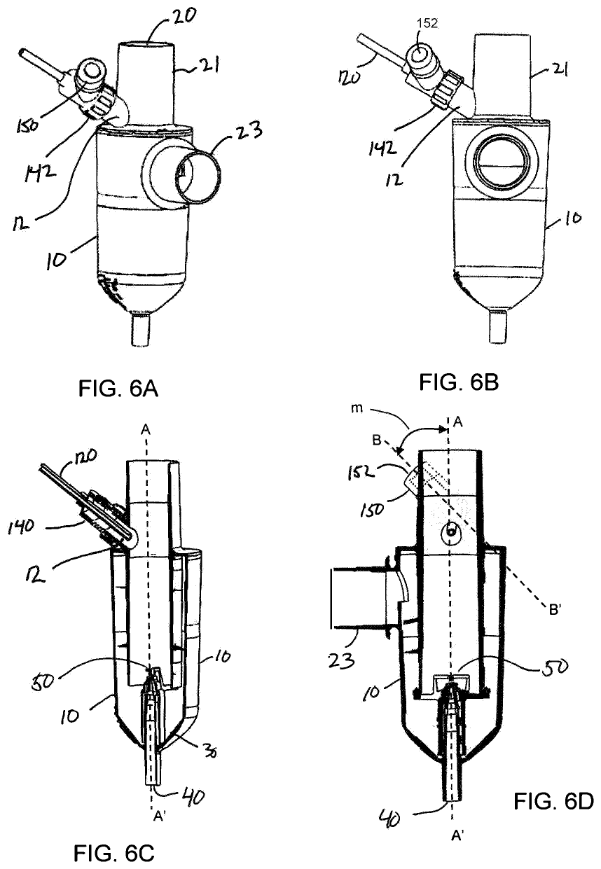

An embodiment of a simple port cap 100 suitable for connection to threads 15 is shown in FIG. 4. The cap 100 is cylindrical with annular wall 110 with proximal lip 112 and distal side 114. As used herein, the terms “proximal” and “distal” are in relation to a longitudinal axis A-A′ (FIG. 6B) through the center of the nebulizer body (e.g., 10). The distal face of 100 comprises a wall (114) with tube 120 traversing through wall 114. Distal wall 114 is has the same configuration as wall 116 in FIG. 5. Tube 120 includes distal end 122 shaped to fit into a plastic tube (180) that may be connected to a syringe or syringe pump (not shown). Proximal end 124 of tube 120 extends into the nebulizer body through a channel defined by port 12 (FIG. 6B). A drug solution can be added the nebulizer 10/11 through tube 120, and the solution will cascade to reservoir 30 for nebulization. In an embodiment, cap 100 has a female threaded interior 118 that can mate with male threads 15 (FIG. 1A).

An alternative embodiment (130) of the port assembly is depicted in FIG. 5. The inventive port assembly incorporates several improvements over the design shown in FIG. 4. The disclosed port assembly 130 has a sleeve 140 with a branch 150 and may include a compression nut 142 on proximal end of sleeve 140 adapted to mate with port threads 15 of the nebulizer 11. The sleeve 140 has a sealed distal end 144 with a wall 116 sealing the distal face of the sleeve 140. A tube 120 traverses longitudinally through the seal 116 from the distal end 144 to the proximal end of the port assembly, allowing a drug solution to be added into the nebulizer through the tube 120 without disassembling the nebulizer or the compression nut 142. Tube 120 has a proximal end 124 that projects into port 12 of the nebulizer (FIG. 6B) and a distal end 122 that may be attached to a plastic tube connected to a syringe or syringe pump for adding a first drug solution to the nebulizer while the nebulizer is in use on a ventilator breathing circuit.

In an embodiment, port assembly 130 has a branch 150 on sleeve 140. Branch 150 defines channel 152. A center line C-C′ of channel 152 is oriented at an approximately 45° (±10°) angle (angle n, FIG. 5C) with respect to distal end 144 of sleeve 140 and an axis through 130 aligned with tube. Channel 152 provides for the addition of a second drug solution into the nebulizer. The additional drug solution may be added as a bolus through channel 152. This may be particularly useful, for example, if a syringe pump with a steady drip is connected via a plastic tube to tube 120, and the clinician determines that an additional drug needs to be added to the nebulizer for inhalation by the patient. An expedient of port assembly 130 is that the syringe pump need not be disturbed during the addition of an additional drug. Thus, this is a safety and labor-saving device while administering complex drug regimens to the patient. With the inventive port assembly, a continuous drug infusion is not interrupted, and the breathing circuit integrity is not compromised when another drug is needed.

In FIG. 6A the inventive port assembly 130 is shown in position attached to a nebulizer 11 through the compression nut 142, which is adapted to mate to male treaded port adapter 15 on the nebulizer 10. When the port assembly 130 is attached to the nebulizer 10 by tightening the compression nut, the orientation of channel (side port) 152 is important and may be as shown in FIGS. 6A and 6C, with an axis B-B′ through the center of port 152 at about a 45° angle with respect to a longitudinal axis through the center of nebulizer body (A-A′). This is shown as angle m in FIG. 6C. In an embodiment, the angle m is between 30° and 80°. Other orientations, for example where 152 is pointing down or up are incorrect. When 152 is pointing at too high of an angle (m near the port 152 may come into conflict with other parts of breathing circuit plumbing (not shown). If angle m is greater than about 80°, a drug solution introduced into 152 will not flow downward or may flow partly out of 152. This configuration allows for a second drug solution (also termed herein an additional drug solution), which can be distinct form the first drug solution, to be added to the nebulizer through the side port 150 along with a first drug solution added to the nebulizer 10 through the tube 120.

FIG. 6B is a cutaway view of a nebulizer 10 with port assembly 130 in position, showing the arrangement of tube 120 and channel 141 in sleeve 140. It can be seen that a first drug can be injected into tube 120 and a second drug can be added to via port 152 that enters the nebulizer body through channel 141. Both drugs will cascade downward to reservoir 30, ready for nebulization in the Venturi section of the nebulizer.

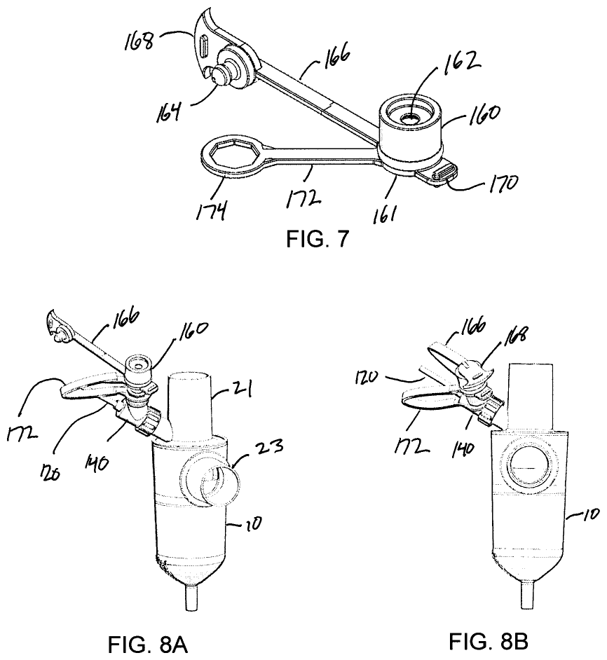

In an embodiment, side port (branch) 150 is equipped with a cap assembly 161 having cap 160, which as depicted in FIG. 7 has an opening 162 permitting the addition of an additional drug solution. The additional drug can be added by syringe or respule through port 162. A tethered plug 164 may be provided that can be inserted into port 162 to seal the opening and prevent air leakage from 162 when the port assembly is not being used to add an additional drug solution to the nebulizer. A tether 166 may be provided securing plug 164 to the entire apparatus, so the plug remains physically attached to nebulizer when it is removed for the addition of a second drug, as shown in FIG. 8A. A finger tab 168 may be provided to make attachment and detachment of plug 164 easier. FIG. 8B shows cap assembly in position with plug 164 inserted into opening 162.

Additionally, a second tether 172 may be provided to keep the cap 160 attached to the nebulizer 10 body, so it remains in physical contact with the nebulizer body even when the cap is detached from branch 150. Also shown is loop 174 on tether 172 that loops over branch 150. Also shown is tab 170 that provides a finger grip for adding or removing assembly 161 from branch 150.

FIG. 8A shows the cap 160 in position on the port assembly 130, where the port assembly is on nebulizer 10. The plug 164 is not engaged in opening 162. This is the position in which a second drug solution can be added to the nebulizer.

FIG. 8B shows the cap 160 in position on the port assembly 130, where the port assembly is on nebulizer 10. In this figure, plug 164 is engaged in opening 162. This is the default position of plug 164 when the nebulizer is in normal use, without a drug solution being added.

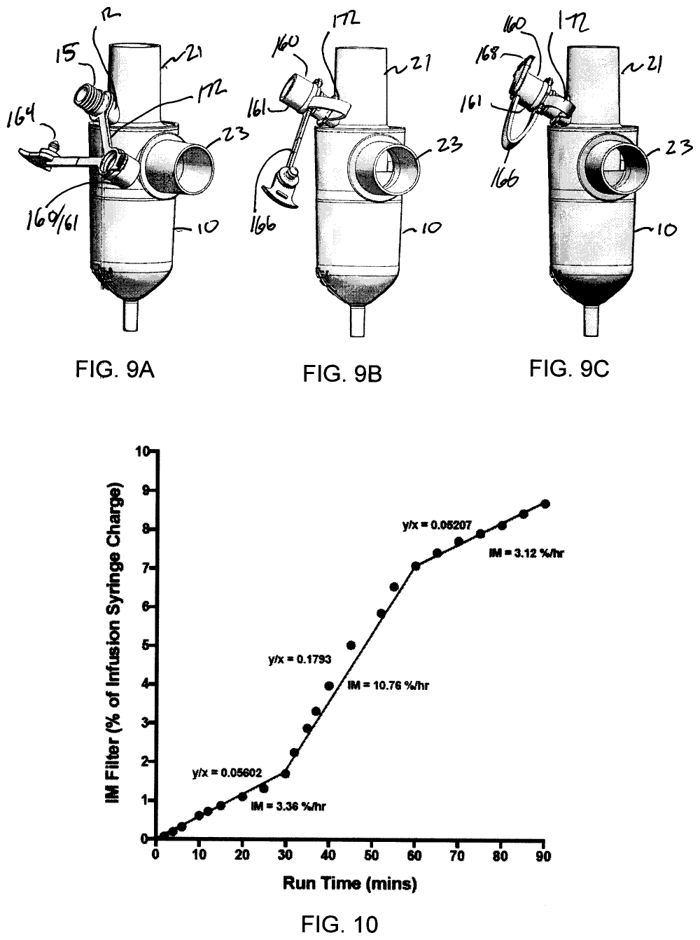

FIGS. 9A-C show an alternative embodiment showing the cap assembly 161 connected to nebulizer 10 without port assembly 130. In this embodiment, cap 160 can fit directly on to threads 15. FIG. 9A shows the assembly 161 with tether 172 connected to the neck of port 12 on the nebulizer. FIG. 9B shows the assembly 161 in position for the addition of a drug solution to the nebulizer through opening 162 (not visible in FIG. 9B). FIG. 9C shows the assembly 161 with plug 164 (not visible) engaged in opening 162. This is the position during normal operation of the nebulizer in the ventilator circuit. The embodiment of FIGS. 9A-C can be used for a continuous infusion of a drug solution into the nebulizer via a syringe. In addition, drug solutions in respules can be added to the nebulizer.

The value of the inventive port 130 is shown in FIG. 10. The data indicates drug delivery to the inhaled mass filter on a ventilator circuit for a duty cycle of 0.13. The “duty cycle” is the portion of an entire breathing cycle when the patient is actually inhaling. The Y axis is inhaled mass as a % of the initial amount of radioactivity in the infusion syringe. The X axis is time in minutes.

In this experiment, 10 mL per hour of a test solution (a first simulated drug) of 53.5 mL of normal saline labeled with 4390 μCi of Technetium 99m pertechnetate (Tc99m) was injected into the nebulizer though port 122 with a syringe pump. The nebulizer was primed with 2 mL of this solution at t=0. At t=30 minutes, a bolus of 3 mL of normal saline mixed with 1213 μCi of Tc99m, a simulated second drug, was injected though opening 162 on cap assembly 161 in position as shown in FIG. 8A. This bolus (second drug) was consumed at t=60 min, but the test solution (first drug) was still being injected at 10 mL per hour until the end of the experiment.

The data shows a linear delivery of the infused simulated drug from 0-30 min, with a slope of about 0.06. When the bolus of the second drug was injected into the nebulizer at 30 minutes, a rapid increase in aerosol delivery was observed (slope=0.18) until the 3 mL of the second drug was consumed, then the underlying continuous infusion of the first drug continues until the end of the experiment, still at a linear drug delivery rate, with a slope of about 0.05. The volume of the bolus giving similar results is not critical. Bolus quantities of 1 mL to 6 mL (the capacity of the drug reservoir in the nebulizer) have been tested with favorable results.

This demonstrates that multiple drugs can be added to the nebulizer while it is in-line in a breathing circuit and linear nebulization rates are obtained. The inventive port allows a first drug to be added at a steady rate, and a second drug can be added through the branch port and be efficiently and linearly delivered to the patient, while leaving the nebulizer in place the entire time.

Some representative drugs that this invention may be useful for include epoprostenol and albuterol which may be added by infusion. Other drugs include gentamicin, tobramycin, and colistin which may be added as a bolus.

Drawings Legend

| | |
|---|---|
| 10 | Jet nebulizer with threaded drug addition port |
| 11 | Jet nebulizer as disclosed in WO2019/236896 A1 |
| 12 | Port in nebulizer for the addition of a drug solution to the nebulizer |
| 14 | Plug for the nebulizer port when not in use |
| 15 | Threads on port 12 |
| 20 | Input airway on nebulizer |
| 21 | Input airway channel |
| 22 | Output airway channel |
| 23 | Output airway on nebulizer |
| 30 | Drug solution reservoir in nebulizer |
| 40 | Compressed air input port on nebulizer |
| 50 | Venturi section in nebulizer |
| 100 | Port cap |
| 110 | Annular wall of port cap |
| 112 | Proximal lip of annular wall |
| 114 | Distal side of port cap (FIG. 4) |
| 116 | Distal wall on port cap (FIG. 5) |
| 118 | Threads on interior of port cap |
| 120 | Drug addition tube |
| 122 | Distal end of drug addition tube |
| 124 | Proximal end of drug addition tube |
| 130 | Port assembly for addition of multiple drugs to the nebulizer |
| 140 | Port sleeve |
| 141 | Channel in sleeve 140 |
| 142 | Compression nut |
| 144 | Distal end of port sleeve |
| 150 | Branch on sleeve |
| 152 | Channel in branch |
| 160 | Cap for branch 150 covering channel 152. |
| 161 | Cap assembly |
| 162 | Opening in cap for addition of second drug solution |
| 164 | Plug for opening 162 |
| 166 | Tether for plug 164 |
| 168 | Finger tab on plug 164 |
| 170 | Finger tab to aid removal of cap |
| 172 | Tether for securing cap 160 to nebulizer |
| 174 | Loop at end of tether 172 for connection to 150 |
| 200 | Ventilator |
| 210 | Inspiratory limb of ventilator circuit |
| 220 | Expiratory limb of ventilator circuit |
| 230 | Humidifier |
| 240 | Air supply for nebulizer |
| 250 | Lungs of patient |

1. A port assembly (130) for the addition of more than one drug solution to a nebulizer (10) having a drug reservoir (30), wherein the nebulizer is used to administer drugs to a patient by inhalation, and the nebulizer is part of a breathing circuit connected a mechanical ventilator, comprising
a. a sleeve (140) having a branch (150), wherein the sleeve has a compression nut (142) at a proximal end adapted to mate with a male threaded port (15) integral with the body of nebulizer (10), wherein the port (15) is adapted as a channel for adding a drug solution to a nebulizer, wherein the male threaded port is on an upper portion of a nebulizer such that a liquid added to the nebulizer through the port will cascade into a drug solution reservoir (30) in the nebulizer;
b. wherein the sleeve (140) has a sealed distal end (116) and a tube (120) traversing longitudinally through the seal from the distal end to the proximal end, wherein a first drug solution can be added into the nebulizer through the tube (120) without disassembling the nebulizer or the compression nut on the sleeve;
c. where the branch (150) on the sleeve defines a channel (152) at an approximately 45° angle with respect to the distal end of an axis defined by tube (120), and the channel provides for the addition of an additional drug solution into the nebulizer;
d. wherein the branch has a cap (160) having an opening (162) therein permitting the addition of the additional drug solution to the nebulizer, wherein the additional drug solution is added by syringe or respule, wherein a plug (164) is provided to prevent air leakage from the opening (162) when the opening is sealed and not in use to add an additional drug solution, and
e. wherein a first drug solution and an additional drug solution can be added simultaneously to the nebulizer.

2. The port assembly of claim 1, wherein the plug (164) is on a tether (166) affixed to the nebulizer such that the plug remains attached to the nebulizer when the plug is removed to expose the opening in the cap for the addition of the additional drug solution.

3. The port assembly of claim 1, wherein an axis through the center of the branch channel (152) is oriented at a 30° to 80° angle from a longitudinal axis though the center of the nebulizer and with respect to the top of the nebulizer.

4. A nebulizer (10) for the administration of a nebulized drug to a patient on a mechanical ventilator, wherein the nebulizer is part of a breathing circuit, comprising a drug input port (12) on the nebulizer allowing a drug solution to be added to the nebulizer without disassembling the nebulizer and without interrupting the breathing circuit, and wherein the input port has male threads (15).

5. A port assembly for the addition of a drug solution to a nebulizer (10) having a drug reservoir (30) and a vertical orientation, comprising a cap (100) with a sealed distal end and a tube (120) traversing longitudinally through the seal from the distal end to a proximal end, wherein a drug solution can be added into the nebulizer through the tube without disassembling the nebulizer, wherein the cap fits over a port (12) adapted as a channel for adding a drug solution to a nebulizer such that a liquid added to the nebulizer through the port will cascade into a drug solution reservoir (30) in the nebulizer.

6. A cap assembly (161) for the addition of a drug solution to a nebulizer (10) having a drug reservoir (30) and a vertical orientation, comprising a cap (160) with an opening (162) therein permitting the addition of an additional drug solution added by syringe or respule to the nebulizer, wherein a plug (164) is provided to prevent air leakage from the opening when the opening is not in use to add an additional drug solution.

7. The cap assembly of claim 6, wherein the cap assembly is connected to the nebulizer body with a tether (172).
